(19) United States
(12) Patent Application Publication
(10) Pub. No.: US 2012/0107232 A1
(43) Pub. Date: May 3, 2012

(54) KIT FOR PREPARING A RADIOLABELED LIPOSOME AND A METHOD USING THE SAME

(75) Inventors: WEI-CHUAN HSU, Taoyuan County (TW); Te-Wei Lee, Taipei City (TW); Shu-Pei Chiu, Taoyuan County (TW); Yi-Ting Tsai, New Taipei City (TW); Gann Ting, Taoyuan County (TW)

(73) Assignee: Institute of Nuclear Energy Research Atomic Energy Council, Executive Yuan, Taoyuan County (TW)

(21) Appl. No.: 13/169,268

(22) Filed: Jun. 27, 2011

(30) Foreign Application Priority Data
Oct. 29, 2010 (TW) 099137268

(51) Int. Cl. A61K 51/12; A61P 35/00

(52) U.S. Cl. 424/1.21

(57) ABSTRACT

A kit for preparing a radiolabeled liposome is provided, the kit including a liposome suspension and a radionuclide, wherein the liposome suspension includes a conjugate with a structure of [chelator-hydrophilic polymer-lipid]. A method for preparing a radiolabeled liposome using the kit is also disclosed herein, thereby the radiolabeled liposome being produced with the conjugate connected to the surface therein. The advantages of the present disclose such as simple, convenient and without purifying for the produced radiolabeled liposome are thus achieved. Further, the produced radiolabeled liposome has a high specific activity and a high sensitivity and suits for the clinical use.

### TECHNICAL FIELD

This disclosure in general relates to preparing radiolabeled liposomes. More particularly, this disclosure is related to a kit, a method and a prepared radiolabeled liposome by chelating a conjugate having a structure of [chelator-hydrophilic polymer-lipid] and a radionuclide.

### BACKGROUND ART

### Description of Related Art

Liposome refer to a lipid bilayer carrier in which is a hydrophilic condition in the interior. This drug delivery system has been shown to significantly change the pharmacokinetics, reduce drug toxicity and thus improve the therapeutic effect. Liposomes (about 30 to 200 nm in size) have been shown that they can pass through the vascular slit in lively angiogenesis for passive targeting accumulation in tissues by the mechanism of EPR (enhanced permeability and retention) effect. Tissues such as infective tissues, inflammatory tissues or tumor tissues are the regions in which liposomes can be specifically accumulated. According such characteristics, radioisotopes are labeled to liposomes in many researches, and thereby developed all kinds of contrast agents for infective tissues, inflammatory tissues or tumor tissues, etc. or for tumor therapeutic drugs.

Generally, methods for labeling radioisotopes to liposomes are divided into two ways. One is the embedding (after loading) method, which is the most common method. For example, Bao et al. published that labeling N,N-bis(2-mercaptoethyl)-N′,N′-diethylethylenediamine (BMEDA) by Rhenium-188 (Re-188), Rhenium-186 (Re-186) and embedding in the liposome were used in basic researches of normal mice for radiotherapy or contrast agents of radiodiagnosis. (Bao et al. J. Pharm. Sci (2003) 92, 1893-1904 and J. Nucl. Med. (2003), 44, 1992-1999)

Further, there are the following two examples for embedding indium-111 (In-111) into the liposome: One is the tumor contrast agent VesCan® (In-111-liposome) in which the In-111 enters into the liposome through the ionophore to bind nitrilotriacetic acid, and thus stably remain within the liposome. After phase III clinical trial, this drug failed to market due to factors such as the drug sensitivity, complexity and competition with other tumor contrast agents, etc. . . .

Recently, the more common method is that: first, In-111 labeled with oxine. Then, the labeled product enters into the liposome to bind with diethylene triamine pentaacetic acid (DTPA) and thus the In-111 can pass through the surface of the liposome and remain within it.

There are still many other related researches, for example, Larsen et al. invented a conjugator system for embedding heavy mental ions into liposomes, wherein the conjugator system comprises liposomes with ionophores and with chelator located inside of the liposomes. (U.S. Pat. No. 6,592,843 B2) In recent years, the method for embedding α-particle emitted radioisotopes into liposomes has been developed. For example, Chang et al. published a method for enhancing Ac-225 to enter and remain in the liposomes (Chang et al. Bioconjugate Chem. 2008, 19, 1274-1282).

There are common disadvantages in using those after loading methods for preparing radiolabeled liposomes:

(1) Radioisotopes need lipidic chelators or ionophores to enter into liposomes. Therefore, labeling of radioisotopes or chelators is necessary for this process.

(2) After entering into liposomes, radioisotopes need reacting with other chelators or buffers to remain stably within liposomes.

(3) Additional purifying steps are required because of the no high loading efficiency (Typically, the better efficiency is about 60% to about 80%).

(4) The specific activity of drugs is low. For example, when the loading efficiency of Re-188-BMEDA-liposome or In-111-oxine-liposome is about 60% to 50%, each liposome only embeds 0.5 to 1.5 radioisotopes.

(5) The whole labeling process is complex, time-consuming. Also, this process wastes materials such as sources or the like and leads to adverse drug production or clinical use.

The other method for labeling radioisotopes to liposomes is surface labeling method but is rarely used now. In the method, radioisotopes label directly with liposomes having chelators on the surface. For example, labeling technetium-99m (Tc-99m) and HYNIC-liposome may be contrast agents for infective tissues or inflammatory tissues. (Peter et al. J. Nucl. Med(1999), 40, 192-197) Comparing Tc-99m-HYNIC-liposome to the conventional Tc-99m-HMPAO-liposome labeled by embedding, the labeling method for Tc-99m-HYNIC-liposome has been found that it may be operated simply and efficiently. Besides, the method may have a better stability and the same characteristics during in vivo experiments.

In recent years, Suna et al. used an amphipathic polychelating compound disclosed in U.S. Pat. No. 5,534,241 in the surface labeling of the liposome to obtain labeled products with high specific activity. The labeled products may used in tumor diagnosis and be very effective. However, binding radioisotopes for chelators of the said reference may be limited and the preparing method thereof may be confined to laboratory use, so the application may be very discommodious. Thus, for clinical application, improvement is required.

### SUMMARY

Abbreviated List

DOTA (1,4,7,10-tetraazacyclotetradecane-N,N′,N″,N′″-tetraacetic acid)

DSPC (distearoyl phosphatidylcholine)

PEG (polyethylene glycol)

DSPE (distearyl phosphatidylethanolamine)

It is therefore a first aspect of the present disclosure to provide a kit for preparing a radiolabeled liposome, which uses a lipid derivative of a bifunctional chelator located on the liposome surface to bind a radioisotope. The operation of the kit may be simple without purifying and significantly reduce costs. Thus, radiolabeled liposome may be prepared rapidly about half-hour at 45° C. to 70° C.

It is a second aspect of the present disclosure to provide a method for preparing a radiolabeled liposome. The method can be widely applied in surface labeling of liposomes. When radiolabeled liposomes synthesize, bifunctional compounds having the structure of [chelator-hydrophilic polymer-lipid] may be introduced simultaneously to synthesize bifunctional chelators having the capacity for directly labeling radioisotopes by the method.

It is therefore a third aspect of the present disclosure to provide a radiolabeled liposome to use easily and apply widely. For example, DOTA is a bifunctional chelator, forming a DOTA-liposome by DSPE-PEG-DOTA.

The DOTA-liposome can label radioisotopes such as indium-111 (In-111), lutetium-177 (Lu-177), gallium-67 (Ga-67), gallium-68 (Ga-68), copper-64 (Cu-64), yttrium-90 (Y-90) or other radionuclides being capable to bind DOTA. Among them, In-111-liposome or Ga-67-liposome can be applied in contrast diagnosis of Single-photon Emission Computed Tomography (SPECT). Ga-68-liposome or Cu-64-liposome can be applied in contrast diagnosis of Positron Emission Tomography (PET), while Lu-177-liposome and Y-90-liposome can be used in malignant tumor treatment.

Thus, the present disclosure provides a kit for preparing a radiolabeled liposome, including:

a liposome suspension dissolved in an aquatic buffer, including the following constituents:
(i) a phopholipid compound selected from the group consisting of lecithins, phosphatidylcholines (PCs), phosphatidylethanolamines (PEs), phosphatidylglycerols (PGs), phosphatidylinositols, sphingomyelins (SMs), phosphatidic acids and the derivatives of the foregoing compounds;
(ii) a cholesterol;
(iii) a phopholipid compound derived from polyethylene glycols (PEGs), wherein the phopholipid may be selected from the group consisting of lecithins, phosphatidylcholines (PCs), phosphatidylethanolamines (PEs), phosphatidylglycerols (PGs), phosphatidylinositols, sphingomyelins (SMs), phosphatidic acids and the derivatives of the foregoing compounds;
(iv) a conjugate having a structure of [chelator-hydrophilic polymer-lipid], wherein the chelator includes at least two binding sites; and

a radionuclide solution in which the radionuclide is capable of chelating with a chelator and is selected from the group consisting of indium-111 (In-111), lutetium-177 (Lu), gallium-67 (Ga-67), gallium-68 (Ga-68), copper-64 (Cu-64), and yttrium-90 (Y-90).

In some embodiments, the liposome suspension may include a plurality of suspending liposome particles, each of which has a mean particle diameter between about 30 and about 200 nm.

In some embodiments, the aquatic buffer may have a pH between about 4 and about 7. In a specific embodiment, the aquatic buffer may be a sodium acetate solution at a concentration between about 0.1 M and about 0.4 M.

In some embodiments, the phopholipid compound of (iii) may be selected from the group consisting of distearyl phosphatidylethanolamine (DSPE), hydrogen soybean phosphotidylcholine (HSPC), egg phosphatidylcholine (EPC) and disteroyl phosphatidylcholine (DSPC), but is not limited to this.

Preferably, the phopholipid compound of (iii) may be selected from the group consisting of DSPE and DSPC in a specific embodiment.

In some embodiments, the phopholipid compound derived from polyethylene glycols (PEGs) of (iii) may be selected from the group consisting of polyethylene glycol-phosphatidylethanolamines (PEG-PEs), methoxypolyethylene glycol-phosphatidylethanolamines (mPEG-PEs) and the derivatives of the foregoing compounds, but is not limited to this.

Preferably, in a specific embodiment, the phopholipid compound derived from PEGs may be mPEG-DSPEs.

In some embodiments, the molar ratio of constituents (i) [symbol] (ii) [symbol] (iii) and (iv) of the liposome suspension may be about 5 to about 10:about 2 to about 10:about 0.1 to about 0.5:about 0.1 to about 0.5. For example, in a specific embodiment, the molar ratio of constituents (i) [symbol] (ii) [symbol] (iii) and (iv) of the liposome suspension is about 3:about 2:about 0.3:about 0.24. in another specific embodiment, the constituents (iii) and (iv) of the liposome suspension is about 0.1 to about 6% of all compounds of the kit.

A method for preparing a radiolabeled liposome is also provided in this disclosure, including the steps of:

(a) Providing any foregoing kit, wherein the kit includes: a liposome suspension including a plurality of suspending liposome particles and a radionuclide solution; and

(b) Pouring the liposome particles of the liposome suspension into the radionuclide solution, fully mixing, and then reacting at least about 10 to 120 minutes to obtain the radiolabeled liposome.

In some embodiments, the preferred reacting temperature of the step (b) may be about 45 to about 70° C., more preferably about 55 to 65° C. and most preferably about 65° C.

The present disclosure also provides a radiolabeled liposome produced by any above-mentioned method, wherein the radiolabeled liposome includes:

a liposome having a surface which includes a conjugate linked to the surface, the conjugate having a structure of [bifunctional chelator-hydrophilic polymer-lipid], wherein the chelator comprises at least two binding sites; and a radionuclide chelated with the chelator, wherein the radionuclide may be selected from the group consisting of indium-111 (In-111), lutetium-177 (Lu-177), gallium-67 (Ga-67), gallium-68 (Ga-68), copper-64 (Cu-64) and yttrium-90 (Y-90).

The term “chelator” herein refers to any bifunctional chelator at least. That is, the chelator may have at least two binding sites in which one site is used for chelating metal ions and the other one is used for coupling liposomes or other ligands.

In some embodiments, the chelator may include but not limit to EDTA, diethylene triamine pentaacetic acid (DTPA), 1,4,7,10-Tetraazacyclotetradecane-N,N′,N″,N′″-Tetraacetic acid (DOTA), nitrotriacetic acid (NTA), deferoxamine, dexrozpxane and the derivatives the foregoing compounds. Preferably, the chelator may be DOTA.

In some embodiments, the hydrophilic polymer may be selected from the group consisting of polyglycine, PEG, polypropylene glycol (PPG), polymethacrylamide, polydimethylacrylamide, poly(hydroxy ethylacrylate), poly(hydroxypropyl methacrylate), polyoxyalkene and hydrophilic peptides, but is not limited to this. That is, any hydrophilic polymer, which may improve biocompatibility, have low toxicity and no charge, and decompose in vivo, can be used.

In a specific embodiment, the hydrophilic polymer may be PEG. The hydrophilic polymer has a preferred average molecular weight of about 100 to 10000 Daltons, more preferably about 100 to 3000 Daltons and most preferably about 2000 Daltons.

The term “lipid” herein refers to any natural or synthetic amphoteric molecule in which has both of the hydrophilic moieties and the hydrophobic moieties to form bilayer vesicles spontaneously, or can be incorporated into lipid bilayer stably. For example, the lipid may be selected from the group consisting of phopholipid, stearylamine, dodecylamine, cetylamine, palmitic acid vinyl ester, 2,3-dihydroxypropyl 12-hydroxy-9-octadecenoate, hexadecyl tetradecanoate, isopropyl myristate, amphoteric acrylic polymer, fatty amide, cholesterol, cholesterol ester, diacylglycerol succinate, diacylglycerol, fatty acid and the derivatives of the foregoing compounds, but is not limited to this.

In some embodiments, phopholipid may be a phopholipid compound of phosphodiglyceride and sphingolipid. In other embodiments, the phopholipid compound preferably selected from the group consisting of lecithins, PCs, PEs, PGs, phosphatidylinositols, SMs, phosphatidic acids and the derivatives of the foregoing compounds, but is not limited to this.

More preferably, the phopholipid compound may be selected from the group consisting of DSPE, HSPC, EPC and DSPC. Most preferably, the phopholipid compound may be selected from the group consisting of DSPE and DSPC.

The details of one or more embodiments of the invention are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 illustrates MADLI/TOF/TOF analysis of DSPE-PEG-DOTA;

FIG. 2 illustrates the result of MicroSPECT/CT qualitative analysis for In-111-DOTA-liposome (A) and In-111-liposome (B) in LS174T tumor-bearing mice; and

FIG. 3 illustrates the schematic drawing of planar γ-imaging quantitative analysis for In-111-DOTA-liposome (A) and In-111-liposome (B) in LS174T tumor-bearing mice.

### DISCLOSURE OF INVENTION

The following examples serve to illustrate certain embodiments and aspects of the present disclosure and are not to be considered as limiting the scope thereof.

### Examples

### 1. Preparation and Quality Control of DSPE-PEG-DOTA

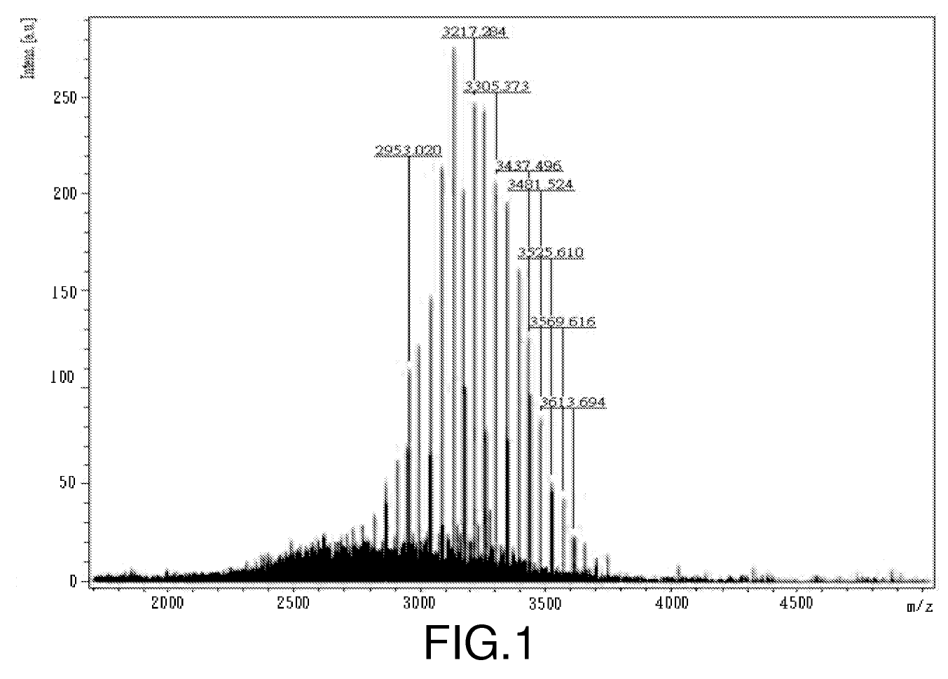

DOTA-NHS-ester and DSPE-PEG₂₀₀₀-NH₂ were weighed respectively (the molar ratio is 2.5:1.), then adding 2 mL dimethylformamide (DMF) to make them fully dissolved. The solution containing DSPE-PEG₂₀₀₀-NH₂/DMF was moved into a 50 ml two-neck bottle in which added 10-fold molar triethylamine (TEA), stirring with a stir bar for one hour. DMF solution of DOTA-NHS-ester was added into the mixture to stir with the stir bar for 24 hours. After the reaction finished, DMF was fully removed by the vacuum system to obtain white solid products. When the solid products were dissolved in the water, they might form micelles. According to this characteristic, the products might be separated by Sephadex Chromatography. The solution which contained the products passed through Sephadex LH-20 gel column, washing by 2% to 14% methanol solution, and then collecting 0.5 mL per column. Then, product positions were determined by the bicinchoninic acid protein assay (the BCA assay) and a portion of pure products were collected while the solvent was removed by the microcontroller freeze dryer. The result of MADLI/TOF/TOF analysis was illustrated in FIG. 1. The average molecular weight is 3217 Da

### 2. Preparation and Quality Control of DOTA-Liposome

DSPC (70 μmole)/Cholesterol/DSPE-PEG₂₀₀₀/DSPE-PEG-DOTA (the molar ratio is 3:2:0.3:0.24) were weighed respectively in a round-bottom flask of 250 mL, then adding 8 mL chloroform to make them fully dissolved. Lipid films formed on the wall of the flask after chloroform was fully removed by the rotary evaporator at about 60° C. in a vacuum. Then, 5 mL of 250 mM ammonium sulfate solution (pH 5.0, 530 mOs) was added in the round-bottom flask in which lipid films had been formed. Multilayer liposomes vesicle (MLV) were obtained by shaking the round-bottom flask in the water bath of 60° C. until all lipid films on the wall dispersed in the ammonium sulfate solution. Further, the suspension which contained multilayer liposomes was repeatedly frozen (in liquid nitrogen) and thawed (in the water bath of 60° C.) six times. After that, single-layer liposomes vesicle were obtained by filtration and extrusion of the LIPEX™ extruder (Lipex Biomembrane, Vancouver, Canada). The suspension which contained single-layer liposomes vesicle passed through Sephadex G50 gel filtration column and eluted with 0.9% NaCl eluting solution. The passed suspension was collected and then dissolved in the 0.9% NaCl buffer to centrifuge at a low-speed. After that, The buffer for the passed suspension was replaced to dissolved in 0.1 M sodium acetate (pH=5.5), The dissolved solution was put into bottle A (1 mL of the volume and 15 μmole/mL of the phospholipid concentration) which was sealed for quality control of the DOTA-liposome:

(1) the normal distribution of an average diameter of 80 nm to 120 nm of the liposome was measured by nano-ZX particle size snalyzer (Malvern, UK.).

(2) the phospholipid concentration in the liposome was measured by the Bartlett's method, the protocol of this method as follows:

Standard solutions and DOTA-liposomes in different concentrations (each 0.5 mL) were provided in test tubes, in which added respectively 10 N H₂SO₄ of 400 μL and reacted for 30 minutes at 180-200° C. in the dry bath, Then, test tubes were taken out and cooled at room temperature. Further, 10% H₂O₂ of 100 μL was added into each test tube to reacted for 30 minutes at 180-200° C. Until solutions in test tubes was clear, test tubes were taken out and cooled at room temperature. Then, 4.6 mL of acidic molybdenum acid solution was added into test tubes and shaken. Later, 100 μL of 15% ascorbic acids was added in test tubes and were taken out after 10 minutes in the water bath at 100° C. to cool at room temperature. The absorbance of samples was measured by the spectrophotometer under a wavelength of 830 nm. According to the linear regression equation which was made by comparing the obtained data from the foregoing method to standard solutions, the content of phosphor was thus calculated.

### 3. Preparation and Quality Control of In-111-DOTA-Liposome

1 mL of DOTA-liposomes in bottle A were taken out with a syringe and injected directly into bottle B which contained about 1-10 μL of ¹¹¹InCl₃ solution which was dissolved in 0.01 N HCl and had a specific activity of 10-300 μCi/μL. After fully shaken in the vortex, bottle B was put in the water bath (60° C. [symbol] 100 rpm) to react more than 30 minutes. After the reaction finished, In-111-DOTA-liposomes were obtained. Partial In-111-DOTA-liposomes were taken for quality control as follows:

(1) the particle size and the phospholipid concentration were determined respectively by the particle size analyzer and the Bartlett's method. Both of the measured values were similar to the result of DOTA-liposomes. That is, the particle size was about 80 nm to 120 nm while the phospholipid concentration was 15 μmole/mL.

(2) In-111-DOTA-liposomes and unlabeled In-111 was separated by the PD-10 column and then calculated the labeling efficiency. The labeling efficiency of In-111-DOTA-liposomes was more than 95%.

(3) the pharmaceutical specific activity (i.e. the capacity for adding radioisotopes with different volume if necessary) was calculated according to the particle size, the phospholipid concentration and the radioactivity. The number of In-111 on each liposome was also calculated and demonstrated that the number of In-111 was up to 13 while the labeling efficiency was more than 95%.

### 4. Preparation and Quality Control of Lu-177-DOTA-Liposome

When preparing Lu-177-DOTA-liposomes, the preparation of bottle A was illustrated in Example 2. But, The buffer for the passed suspension was replaced with 0.2 sodium acetate buffer (pH=4.8) and bottle B was changed as a 0.5 to 5 μL ¹⁷⁷LuCl₃ solution which was dissolved in 0.05 N HCl and had a specific activity of about 600 μCi/μL. Other labeling and quality control methods were identical to Example 3. The labeling efficiency of Lu-177-DOTA-liposomes was more than 95%.

### 5. In vitro Stability Analysis for In-111-DOTA-Liposome

In vitro stability analysis for labeled In-111-DOTA-liposomes in Example 3 was as follows. In-111-DOTA-liposomes mixed respectively with the saline (1:1), the rat serum (1:19) and the human serum (1:19). The mixtures kept at 37° C. and were taken out to analyze after 1, 4, 8, 24, 48, 72 hours, respectively. For preparing the analytical column, Sepharose 4 Fast Flow (GE Healthcare) was filled with Poly-Prep column (Bio-Rad) and then was balanced by the saline. Samples which were taken out at different time eluted with the saline. In-111-DOTA-liposomes were washed out first because of the larger size, and then were counted by the Gamma counter to calculate the ratio In-111-DOTA-liposomes at different time. The result was shown in Table 1 in which In-111-DOTA-liposomes were found to have a stability more than 85% whether in the saline, the rat serum, or the human serum.

TABLE 1
The result of in vitro stability analysis for In-111-DOTA-liposome

| Incubation | Normal saline | Rat plasma | Human plasma |
|---|---|---|---|
| time (hr) | (%) | (%) | (%) |
| 1 | 94.07 ± 0.54 | 94.37 ± 0.79 | 91.14 ± 0.94 |
| 4 | 92.85 ± 0.84 | 96.30 ± 0.70 | 92.38 ± 1.03 |
| 8 | 92.45 ± 1.15 | 95.87 ± 0.52 | 93.57 ± 2.11 |
| 24 | 95.50 ± 0.57 | 93.45 ± 2.23 | 90.23 ± 1.81 |
| 48 | 94.18 ± 0.64 | 89.08 ± 2.63 | 84.46 ± 4.27 |
| 72 | 94.85 ± 0.85 | 87.98 ± 1.02 | 85.26 ± 3.02 |

(mean ± S.D., n = 3)

### 6. Analysis of MicroSPECT/CT Imaging

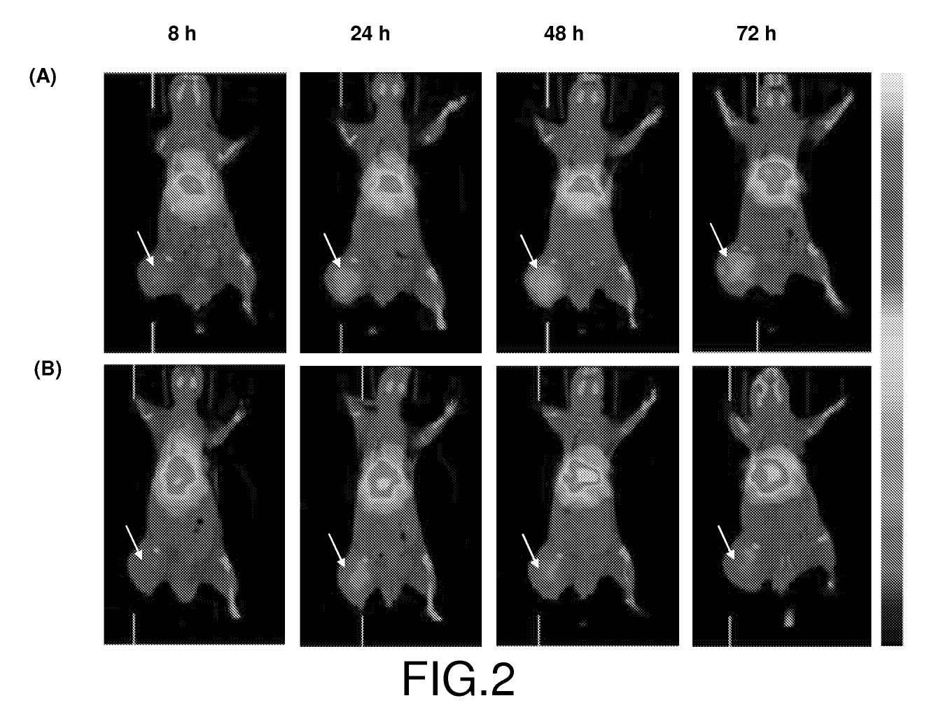

In-111-DOTA-liposomes were under in vivo imaging analysis in which animal models were prepared as follows: 2×10⁵ of LS174T cells (human intestinal cancer cell lines) were subcutaneously injected into the thigh outside of 5 to 6-week-old nude mice. Until the tumor grew 3 to 4 weeks, the microSPECT/CT imaging analysis began. To compare with the conventional labeling method, In-111-liposomes labeled by the In-oxine method were control groups. In-111-DOTA-liposomes and In-111-liposomes were labeled respectively. After the quality control analysis, both labeled products (150 μL in volume, 0.3 mCi/mL of the specific activity, 1.8×10¹² of the injected liposome number, 90.5±24.73 nm of the particle size) were intravenously injected into tumor-bearing mice. After administering respectively at 8th hour, 24th hour, 48th hour and 72th hour, the imaging process of microSPECT/CT proceeded. After the image reconstruction and fusion, the qualitative result was illustrated in FIG. 2. The result demonstrated that both labeled products had an apparent tumor absorption under the tumor animal model, wherein the peak of the absorption reached after administering for 48 hours. The image reconstruction and fusion of the coronal section was shown in FIG. 2.

### 7. Image Quantitative Analysis

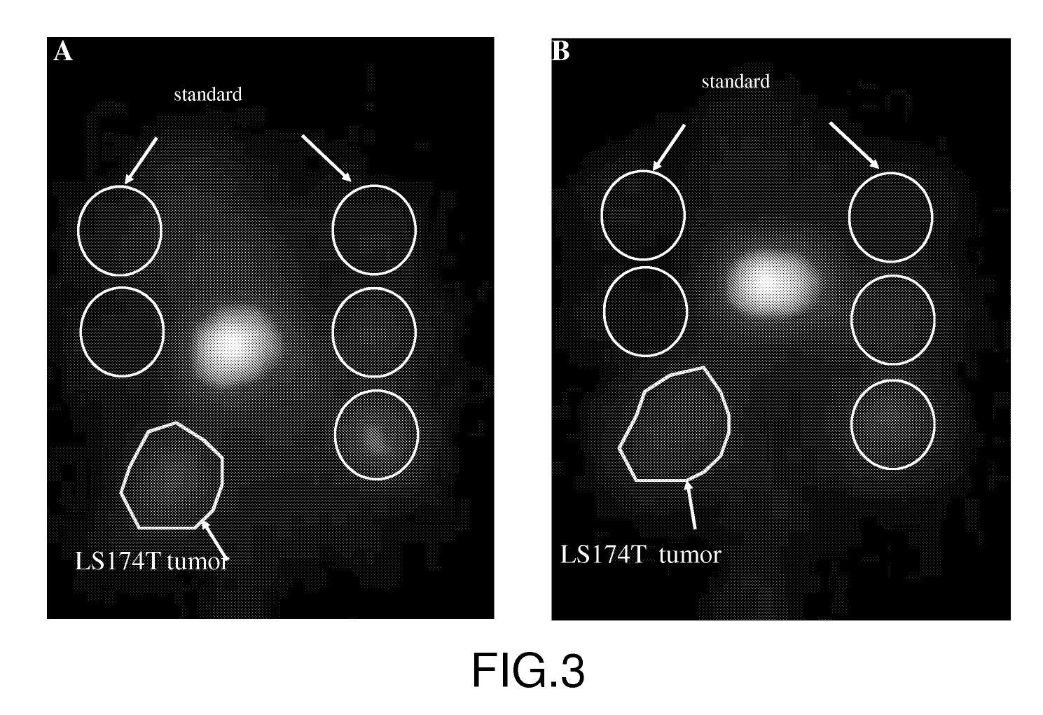

Tumor animal models were illustrated in example 6. Six tumor-bearing mice were randomly divided into two groups and respectively administered as example 6 for 48 hours for Planar Gamma imaging analysis. While imaging, the standard source (i.e. 1, 2, 4, 8 and 16% of the total amount of radiation injected into nude mice) also imaging at the time. The real absorption value (in units of μCi) after administering for 48 hours in the tumor was obtained by choosing the region of interest (ROI) value in the tumor and comparing to the standard source. The real absorption value then divided by the species activity injected into nude mice's body to get % ID. % ID further divided by tumor weight of each nude, which tumor tissues were weighed until nude mice sacrificed after the contrast process finished, and % ID/g (i.e. pharmaceutical absorption ratio per tumor tissue) thus obtained. The result in FIG. 3 was shown that both In-111-DOTA-liposomes and In-111-liposomes had a similar absorption ratio under the animal model of LS174T tumor-bearing mice (P>0.05). This result demonstrated that the labeling method for In-111-DOTA-liposomes did not affect in vivo pharmaceutical stability.

TABLE 2
The result of γ-imaging quantitative analysis for In-111-DOTA-
liposome (A) and In-111-liposome (B) in LS174T tumor-bearing mice.

| | Average tumor | | |
|---|---|---|---|
| Drugs | weight | % ID | % ID/g |
| 111In-DOTA-liposome | 1.56 ± 0.05 | 16.6 ± 1.95 | 10.6 ± 0.91 |
| (n = 3) | | | |
| 111In-liposome | 1.17 ± 0.73 | 13.0 ± 0.45 | 11.1 ± 0.33 |
| (n = 3) | | | |

In conclusion, the present disclosure does provide a kit for preparing a radiolabeled liposome, there are several advantages such as easy use, simple operation, no need for purification, high specific activity and high sensitivity for using the kit with the labeling method of the present disclosure. Further, the prepared radiolabeled liposome may use as a composition binding to indium-111 (In-111), lutetium-177 (Lu-177), gallium-67 (Ga-67), gallium-68 (Ga-68), copper-64 (Cu-64), yttrium-90 (Y-90) and the like. Thus, the prepared radiolabeled liposome may apply in the diagnosis, treatment, PET and SPECT contrast diagnosis and suit to the clinical use.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A kit for preparing a radiolabeled liposome, comprising:
a liposome suspension dissolved in an aquatic buffer, including the following constituents:
(i) a phopholipid compound selected from the group consisting of lecithins, phosphatidylcholines (PCs), phosphatidylethanolamines (PEs), phosphatidylglycerols (PGs), phosphatidylinositols, sphingomyelins (SMs), phosphatidic acids and the derivatives of the foregoing compounds;
(ii) a cholesterol;
(iii) a phopholipid compound derived from polyethylene glycols (PEGs), wherein the phopholipid may be selected from the group consisting of lecithins, phosphatidylcholines (PCs), phosphatidylethanolamines (PEs), phosphatidylglycerols (PGs), phosphatidylinositols, sphingomyelins (SMs), phosphatidic acids and the derivatives of the foregoing compounds;
(iv) a conjugate having a structure of [chelator-hydrophilic polymer-lipid], wherein the chelator includes at least two binding sites; and
a radionuclide solution in which the radionuclide is capable of chelating with a chelator and is selected from the group consisting of indium-111 (In-111), lutetium-177 (Lu-117), gallium-67 (Ga-67), gallium-68 (Ga-68), copper-64 (Cu-64), and yttrium-90 (Y-90).

2. The kit of claim 1, wherein the liposome suspension comprises a plurality of suspending liposome particles, each of which has a mean particle diameter between about 30 and about 200 nm.

3. The kit of claim 1, wherein the aquatic buffer has a pH between about 4 and about 7.

4. The kit of claim 3, wherein the aquatic buffer refers to a sodium acetate solution at a concentration between about 0.1 M and about 0.4 M.

5. The kit of claim 1, wherein the phopholipid compound of (iii) is selected from the group consisting of distearyl phosphatidylethanolamine (DSPE), hydrogen soybean phosphotidylcholine (HSPC), egg phosphatidylcholine (EPC) and disteroyl phosphatidylcholine (DSPC).

6. The kit of claim 1, wherein the phopholipid compound derived from polyethylene glycols (PEGs) of (iii) is selected from the group consisting of polyethylene glycol-phosphatidylethanolamines (PEG-PEs), methoxypolyethylene glycol-phosphatidylethanolamines (mPEG-PEs) and the derivatives thereof.

7. The kit of claim 1, wherein the phopholipid compound derived from polyethylene glycols (PEGs) refers to methoxypolyethylene glycol-distearyl phosphatidylethanolamines. The kit of claim 1, wherein the phopholipid compound derived from polyethylene glycols (PEGs) refers to methoxypolyethylene glycol-distearyl phosphatidylethanolamines (mPEG-DSPEs).

8. The kit of claim 1, wherein the molar ratio of constituents (i) [symbol] (ii) [symbol] (iii) and (iv) of the liposome suspension is about 5 to about 10:about 2 to about 10:about 0.1 to about 0.5:about 0.1 to about 0.5.

9. The kit of claim 1, wherein the constituents (iii) and (iv) of the liposome suspension is about 0.1 to about 6% of all compounds of the kit.

10. A method for preparing a radiolabeled liposome, comprising the steps of:
providing a kit comprises a liposome suspension dissolved in an aquatic buffer, including the following constituents:
(i) a phopholipid compound selected from the group consisting of lecithins, phosphatidylcholines (PCs), phosphatidylethanolamines (PEs), phosphatidylglycerols (PGs), phosphatidylinositols, sphingomyelins (SMs), phosphatidic acids and the derivatives of the foregoing compounds;
(ii) a cholesterol;
(iii) a phopholipid compound derived from polyethylene glycols (PEGs), wherein the phopholipid may be selected from the group consisting of lecithins, phosphatidylcholines (PCs), phosphatidylethanolamines (PEs), phosphatidylglycerols (PGs), phosphatidylinositols, sphingomyelins (SMs), phosphatidic acids and the derivatives of the foregoing compounds;
(iv) a conjugate having a structure of [chelator-hydrophilic polymer-lipid], wherein the chelator includes at least two binding sites; and
a radionuclide solution in which the radionuclide is capable of chelating with a chelator and is selected from the group consisting of indium-111 (In-111), lutetium-177 (Lu-117), gallium-67 (Ga-67), gallium-68 (Ga-68), copper-64 (Cu-64), and yttrium-90 (Y-90); and
(b) pouring the liposome particles of the liposome suspension into the radionuclide solution, fully mixing, and then reacting at least about 10 to about 120 minutes to obtain the radiolabeled liposome.

11. The method of claim 10, wherein the reacting temperature of the step (b) is about 45 to about 70□.

12. A radiolabeled liposome produced by the method of claim 10, comprising:
a liposome having a surface which comprises a conjugate linked to the surface, the conjugate having a structure of [bifunctional chelator-hydrophilic polymer-lipid], wherein the chelator comprises at least two binding sites; and
a radionuclide chelated with the chelator, wherein the radionuclide is selected from the group consisting of indium-111 (In-111), lutetium-177 (Lu-177), gallium-67 (Ga-67), gallium-68 (Ga-68), copper-64 (Cu-64) and yttrium-90 (Y-90).

13. The radiolabeled liposome of claim 12, wherein the chelator is selected from the group consisting of EDTA, diethylene triamine pentaacetic acid (DTPA), 1,4,7,10-Tetraazacyclotetradecane-N,N′,N″,N′″-Tetraacetic acid (DOTA), nitrotriacetic acid (NTA), deferoxamine, dexrozpxane and the derivatives thereof.

14. The radiolabeled liposome of claim 12, wherein the hydrophilic polymer is selected from the group consisting of polyglycine, PEG, polypropylene glycol (PPG), polymethacrylamide, polydimethylacrylamide, poly(hydroxy ethylacrylate), poly(hydroxypropyl methacrylate), polyoxyalkene, hydrophilic peptides, and the derivatives thereof.

15. The radiolabeled liposome of claim 12, wherein the lipid is selected from the group consisting of phopholipid, stearylamine, dodecylamine, cetylamine, palmitic acid vinyl ester, 2,3-dihydroxypropyl 12-hydroxy-9-octadecenoate, hexadecyl tetradecanoate, isopropyl myristate, amphoteric acrylic polymer, fatty amide, cholesterol, cholesterol ester, diacylglycerol succinate, diacylglycerol, fatty acid and the derivatives thereof.

16. The radiolabeled liposome of claim 15, wherein the phopholipid refers to a phopholipid compound comprising a phosphodiglyceride and a sphingolipid.

17. The radiolabeled liposome of claim 16, wherein the phopholipid compound is selected from the group consisting of lecithins, phosphatidylcholines (PCs), phosphatidylethanolamines (PEs), phosphatidylglycerols (PGs), phosphatidylinositols, sphingomyelins (SMs), phosphatidic acids and the derivatives thereof.

18. The radiolabeled liposome of claim 17, wherein the phopholipid compound is selected from the group consisting of distearyl phosphatidylethanolamine (DSPE), hydrogen soybean phosphotidylcholine (HSPC), egg phosphatidylcholine (EPC) and disteroyl phosphatidylcholine (DSPC).
